(19) United States
(12) Patent Application Publication
(10) Pub. No.: US 2026/0236525 A1
(43) Pub. Date: Aug. 13, 2026

(54) RETINA IMAGE REFERENCE DATABASE

(71) Applicant: IMMERSIX LTD, Tel Aviv (IL)

(72) Inventors: Ori WEITZ, Tel Aviv (IL); Haim PERSKI, Hod Hasharon (IL)

(21) Appl. No.: 19/154,383

(22) PCT Filed: Feb. 11, 2024
(86) PCT No.: PCT/IL2024/050159

§ 371 Date: Aug. 7, 2025

(30) Foreign Application Priority Data
Feb. 9, 2023 (IL) 300551

(51) Int. Cl. G06F 16/51; G06F 3/01; G06T 3/4038; G06T 7/73

(52) U.S. Cl. CPC G06F 16/51; G06F 3/013; G06T 3/4038; G06T 7/74; G06T 2200/24; G06T 2207/30201

(57) ABSTRACT

Methods and systems for maintaining a reference database of images of a person's retina, include obtaining a plurality of reference images of the person's retina while the person is assumed to be looking at a known gaze target, each of the reference images capturing a different portion of the retina, and calculating a gaze target associated with a certain reference image from the plurality of reference images. If the calculated gaze target is determined to be the known gaze target, then a command is generated to store image data related to the certain reference image in a reference database used as a reference for tracking gaze of the person. If the calculated gaze target is not determined to be the known gaze target, then the image data related to the certain reference image is not stored in the reference database or it is removed from the reference database.

### FIELD

The present invention relates to gaze tracking based on images of a person's retina.

### BACKGROUND

Eye tracking to determine direction of gaze (also referred to as gaze tracking) may be useful in different fields, including human-machine interaction control of devices such as industrial machines, in aviation, and emergency room situations where both hands are needed for tasks other than operation of a computer, in virtual, augmented or extended reality applications, in computer games, in entertainment applications and also in research, to better understand subjects' behavior and visual processes. In fact, gaze tracking methods can be used in all the ways that people use their eyes.

Some video-based eye trackers use features of the eye, such as, corneal reflection, center of the pupil of the eye and features from inside the eye, such as the retinal blood vessels, as features from which to reconstruct the optical axis of the eye and/or as features to track in order to measure movement of the eye.

In retinal image-based tracking systems, in order to obtain information on a user's eye properties and on the relationship between the user's eye properties and the user's direction of gaze, users are typically asked to look at several known gaze targets, so that images of the eye, when gazing at the known targets, can be recorded and mapped to the known target, thereby creating a reference database of the user, for future use. However, if the user does not look at the known targets, as requested (but, instead, looks at other targets), images of the user's eyes will be falsely mapped to the known target. The system has no indication that the images are falsely mapped, such that the system may be operating ineffectively, based on a defective and inaccurate reference database.

### SUMMARY

Embodiments of the invention provide a system and method for automatically detecting a false image of a person's retina and keeping false images out of a reference database, thereby maintaining a high-quality reference database, enabling accurate and uninterrupted gaze tracking.

In one embodiment of the invention, a method for maintaining a reference database for gaze tracking based on images of the retina, includes obtaining a plurality of reference images of a person's retina, the reference images captured while the person is assumed to be looking at a known gaze target. Each of the reference images captures a different portion of the retina. Image data related to the reference images may be stored in association with the known gaze target, thereby creating a reference database. The method includes determining if a certain reference image is a false image. A false image may be a falsely mapped image of a person's retina (e.g., a reference image captured when the person was looking at a gaze target other than the known gaze target) or a reference image for which it is not or cannot be determined that the gaze target associated with the image, is the known gaze target.

The image data related to the false image is not stored in the reference database, e.g., the image data related to the false image may be removed or deleted prior to being stored or after being stored in the reference database.

### BRIEF DESCRIPTION OF THE FIGURES

The invention will now be described in relation to certain examples and embodiments with reference to the following illustrative figures so that it may be more fully understood. In the drawings:

FIGS. 1A-B schematically illustrate examples of systems for maintaining a reference image database for gaze tracking, according to embodiments of the invention;

FIGS. 2A-B schematically illustrate methods for collecting reference images of a person's retina, according to embodiments of the invention;

FIG. 3 schematically illustrates a method for maintaining a reference database of images of a person's retina, according to embodiments of the invention; and

FIG. 4 schematically illustrates a method for detecting a false image, according to an embodiment of the invention.

### DETAILED DESCRIPTION

Embodiments of the invention provide systems and methods used for gaze tracking based on images of a person's retina, specifically for maintaining a reference image database for gaze tracking. Images of a person's retina typically include features of the retina, such as, patterns of blood vessels that supply the retina. In some cases, images of the retina include the fovea, which is a small depression on the retina. When an image of the world is formed on the retina, an image of the gaze target is formed on the fovea. That is, the location of the fovea corresponds to the direction of gaze of the person.

In some cases (e.g., while enrolling a new user to a gaze tracking system), the person's retina is imaged while the person is looking at a known gaze target. In one embodiment, the person's retina is imaged while the person is looking with continuous eye movement at a single known gaze target, however, due to movement of the target and/or movement of the person's head (while keeping the gaze on the target), a wide portion of the retina can be imaged in a quick and convenient manner.

A ray of gaze corresponding to the person's gaze includes the origin of the ray and its direction. The origin of the ray can be assumed to be at the optical center of the person's eye lens whereas the direction of the ray is determined by the line connecting the origin of the ray and the gaze target. The direction of the ray of gaze is derived from the orientation of the eye.

A change in orientation of a person's eye from one gaze target to another can be calculated based on comparing to each other different images of the person's retina associated with different gaze targets. Thus, a change in orientation of a person's eye between a known gaze target and an unknown gaze target can be calculated by comparing an image of the person's retina associated with an unknown gaze target to a reference image, namely, an image of the person's retina which is associated with a known gaze target.

Embodiments of the invention provide systems and methods for collecting reference images of a person's retina and maintaining the reference images in a reference database. “Reference images” include images or features from images, or image data (as further described below) of different portions of a retina of a specific eye of a specific person (also termed “user”), all associated with a known gaze target (i.e., a known direction of gaze of the specific person). So, essentially, reference images provide retinal features to visually identify a region of a person's retina at a later time, and the relative orientation of the person's visual axis relative to these features.

Systems and methods according to embodiments of the invention, are exemplified below.

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well known features may be omitted or simplified in order not to obscure the present invention.

Unless specifically stated otherwise, as apparent from the following discussions, it is appreciated that throughout the specification discussions utilizing terms such as “analyzing”, “processing”, “computing”, “calculating”, “comparing”, “determining”, “detecting”, “identifying”, “creating”, “producing”, “controlling”, “tracking”, “choosing”, or the like, refer to the action and/or processes of a computer or computing system, or similar electronic computing device, that manipulates and/or transforms data represented as physical, such as electronic, quantities within the computing system's registers and/or memories into other data similarly represented as physical quantities within the computing system's memories, registers or other such information storage, transmission or display devices. Unless otherwise stated, these terms refer to automatic action of a processor, independent of and without any actions of a human operator.

One embodiment of the invention provides a gaze tracking system that includes a camera to capture images of a person's retina while the person is assumed to be looking at a known gaze target. The system also includes a reference database to store image data related to images the person's retina in association with the known gaze target. The system further includes a processor to calculate a gaze target associated with a certain image from the images of the person's retina. If the calculated gaze target is determined to be the known gaze target, then image data related to the certain image is stored in the reference database. If the calculated gaze target is not determined to be the known gaze target, then the certain image is determined to be a false image and the image data related to the certain image is not stored in the reference database. For example, the processor may cause the false image to be removed and/or deleted, e.g., from the reference database.

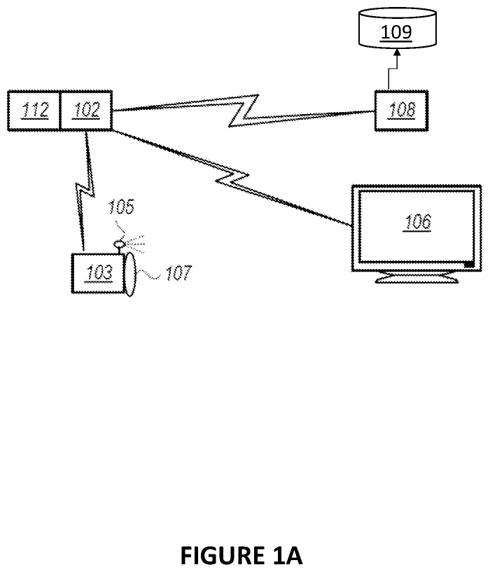

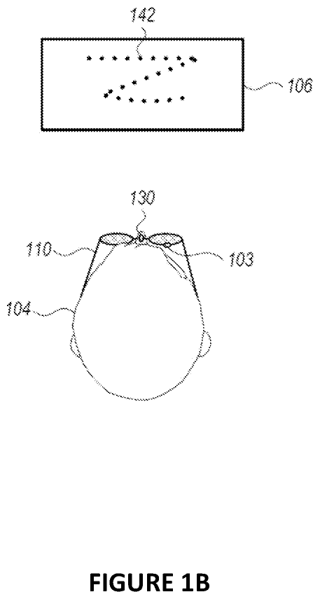

In one embodiment, which is schematically illustrated in FIG. 1A, a gaze tracking system configured to capture images of a person's retina includes one or more camera 103 for capturing images of a person's retina while the person is assumed to be looking at a known gaze target. The system may further include a user interface (UI) device 106 configured to display a target at a known location or locations, for the person to look at. The location of the target is typically at a known location in relation to a frame of reference, for example, to a frame of reference of the display of the UI device 106 or the frame of reference of camera 103.

The person may be looking at the gaze target with continuous or discontinuous eye movement. Continuous eye movement while looking at a target, typically requires keeping an eye (or eyes) on the target. If the target moves, the eye rotates so that it can keep on the target while the target changes locations, enabling a camera to capture a wide area of the retina. A wide range of angles of rays of gaze can be provided and a wide area of the retina can be captured by the camera even if the target does not move, by keeping the eye on the target but moving the head (e.g., back and forth or in a circle). Motion of the head while gazing at a single unmoving target, which changes the angle of ray of gaze, rotates the eye relative to the head. Thus, if camera 103 is connected to the user's head (e.g., if camera 103 is located on a head mounted device, such as glasses, as illustrated in FIG. 1B), motion of the head rotates the eye relative to camera 103, which enables capturing images of many different portions of the retina. The rotation of the eye while tracking a moving target or when moving the head while the gaze is fixed on a single unmoving target, thus enables obtaining images of different portions of the retina that can be saved in association with a known gaze target in a reference database.

UI device 106 may include a display, such as a monitor or screen, for displaying targets and instructions and/or notifications to a user (e.g., via text or other content displayed on the monitor).

A processor 102, which is in communication with the camera 103 and UI device 106, can cause a gaze target to be displayed at a known location (or locations) on a display of UI device 106 and can cause the gaze target to move on the display. In some embodiments, processor 102 can cause instructions to be displayed on the display of UI device 106, for a user to keep the user's gaze on a moving target and/or to move the user's head while keeping the gaze on an unmoving target.

In one embodiment, which is schematically illustrated in FIG. 1B, processor 102 causes a target 142 to move on a display of UI device 106. Target 142 may be moved continuously or not, in a predetermined pattern or in a random pattern.

The system may include an XR device such as XR glasses 110, which are operative based on reference database 109. For example, XR glasses 110 may include a gaze tracking system which uses reference database 109 to calculate a change in orientation of an eye of a user and possibly determine the person's direction of gaze, as described herein. An XR device may include augmented reality (AR), virtual reality (VR) or mixed reality (MR) optical systems, such as Lumus™ DK-Vision, Microsoft™ Hololens, or Magic Leap One™.

A front-facing “world camera” 130 that captures images of the world, may be attached to XR glasses 110.

The display of UI device 106 may be part of an XR device, such as XR glasses 110. In some embodiments, UI device 106 may include markings that will be visible in images captured by world camera 130 and which can mark the edges of the display of UI device 106.

Camera 103 may also be attached to XR glasses 110. Camera 103 can obtain an image of a portion of the person's retina, via the pupil of the eye, with minimal interference or limitation of the person's field of view. For example, camera 103 may be located at the periphery of a person's eye (e.g., below, above or at one side of the eye) a couple of centimeters from the eye.

In some embodiments, processor 102 may also be attached to XR glasses 110.

Referring back to FIG. 1A, camera 103 may include a CCD or CMOS or other appropriate image sensor. Camera 103 images the retina by converting rays of light from a particular point of the retina to a pixel on the camera sensor. Camera 103 may include an optical system which may include a lens 107 and possibly additional optics such as mirrors, filters, beam splitters and polarizers.

In some embodiments, lens 107 has a wide depth of field or an adjustable focus. In some embodiments lens 107 may be a multi-element lens.

The system may include one or more light source(s) 105 configured to illuminate the person's eye. Light source 105 may include one or multiple illumination sources and may be arranged, for example, as a circular array of LEDs surrounding the camera 103 and/or lens 107. Light source 105 may illuminate at a wavelength which is undetected by a human eye (and therefore unobtrusive), for example, light source 105 may include an IR LED or other appropriate IR illumination source. The wavelength of the light source (e.g., the wavelength of each individual LED in the light source), may be chosen so as to maximize the contrast of features in the retina and to obtain an image rich with details. In some embodiments, light source 105 may include a miniature light source which may be positioned in close proximity to the camera lens 107, e.g., in front of the lens, on the camera sensor (behind the lens) or inside the lens.

Processor 102 may be in communication with light source 105 to control, for example, the intensity and/or timing of illumination, e.g., to be synchronized with operation of camera 103. Different LEDs, having different wavelengths can be turned on or off to obtain different wavelength illumination. In one example, the amount of light emitted by light source 105 can be adjusted by processor 102 based on the brightness of the captured image. In another example, light source 105 can be controlled to emit different wavelength lights such that different frames can capture the retina at different wavelengths, and thereby capture more detail. In yet another example, light source 105 can be synchronized with the camera 103 shutter. In some embodiments, short bursts of very bright light can be emitted by light source 105 to prevent motion blur, rolling shutter effect, or reduce overall power consumption.

Processor 102 may receive image data from camera 103 based on which processor 102 can control light source 105 (e.g., as described above). In additional embodiments, processor 102 receives image data from camera 103 and may calculate a change in orientation of an eye of a person (e.g., a user), and possibly determine the person's direction of gaze, based on the received image data.

Image data may include representations of the image as well as partial or full images or videos of the retina or portions of the retina. Representations of the image may include, for example, image information (information describing the image), e.g., key features detailing a location of one or more pixels and information about the image at that location. Information about the image may include, for example, pixel values that represent the intensity of light reflected from a person's retina, a histogram of gradients around the location of the pixel(s), a blood vessel between two points having a given thickness, a blotch described by an ellipse, etc.

Processor 102, which may be locally embedded or remote, may include, for example, one or more processing units including a central processing unit (CPU), a graphics processing unit (GPU), a digital signal processor (DSP), a field-programmable gate array (FPGA), a microprocessor, a controller, a chip, a microchip, an integrated circuit (IC), or any other suitable multi-purpose or specific processing or controlling unit.

In some embodiments, processor 102 may be in communication with a storage device 108 such as a server, including, for example, volatile and/or non-volatile storage media, such as a hard disk drive (HDD) or solid-state drive (SSD). Storage device 108, which may be connected locally or remotely, e.g., in the cloud, may store and allow processor 102 access to a reference database 109, namely, a database of reference images and maps (e.g., lookup tables) linking image data of the reference images with gaze targets.

Database 109 may be used for calculating a gaze target of the person. In some embodiments, processor 102 uses image data related to reference images from reference database 109 to calculate an unknown location of a gaze target by comparing information from reference images (e.g., images of the person's retina associated with a known location), with information from images of the person's retina while the person is looking at a target at an unknown location. Processor 102 may use the calculated unknown target or a signal generated based on the calculated unknown gaze target, to control a device, e.g., as further described below.

To maintain integrity and high quality of the reference database 109, processor 102 may determine that a certain reference image is a false image, e.g., if a calculated gaze target associated with the certain reference image is not (e.g., is not similar to) the known gaze target. The certain reference image may be determined to be a false image prior to being stored in database 109 or after being stored in reference database 109. If a false image is detected prior to being stored in reference database 109, processor 102 may prevent the image from being stored in the reference database. If a false image is detected after it has been stored in the reference database 109, processor 102 may cause the false image to be deleted or removed from the reference database 109.

Components of the system may be in wired or wireless communication and may include suitable ports and/or network hubs.

Processor 102 is typically in communication with a memory unit 112, which may store at least part of the image data received from camera(s) 103. Memory unit 112 may be locally embedded or remote. Memory unit 112 may include, for example, a random access memory (RAM), a dynamic RAM (DRAM), a flash memory, a volatile memory, a non-volatile memory, a cache memory, a buffer, a short term memory unit, a long term memory unit, or other suitable memory units or storage units.

In some embodiments, the memory unit 112 stores executable instructions that, when executed by processor 102, facilitate performance of operations of processor 102, as described herein.

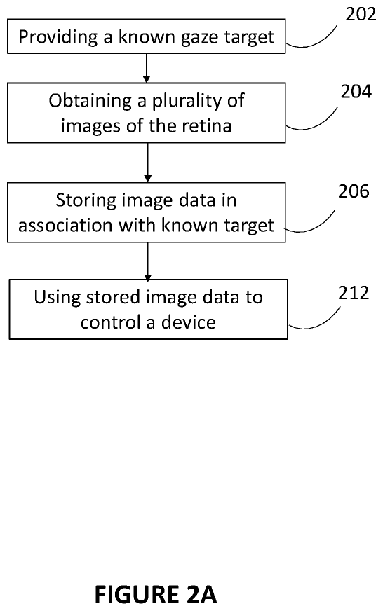

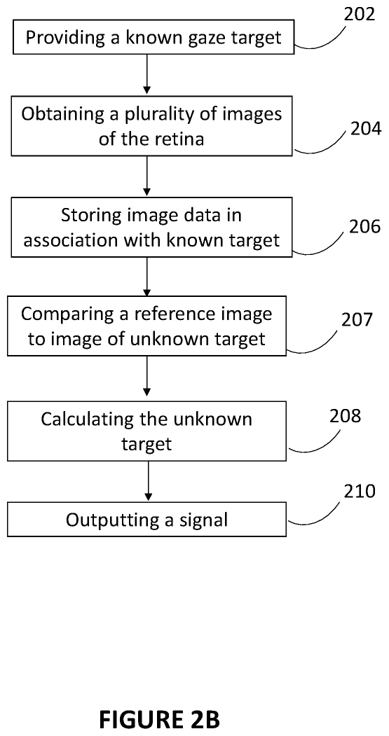

In one embodiment, which is schematically illustrated in FIG. 2A, a method for collecting reference images of a person's retina, is performed by processor 102 and includes providing a known gaze target for a person to look at (202). A “known gaze target” is a target located at a known location, typically a known location in relation to a frame of reference e.g., as described herein and/or at a known direction of gaze of a user. While the person is looking at the known gaze target, a plurality of images of the person's retina are obtained (204), each of the images capturing a different portion of the retina. Typically, at least some of the images—at least partially—overlap.

Image data from the plurality of images is stored in association with the known gaze target in a reference database (206). For example, image data may be associated with a known target gaze (e.g., with a location of the target, typically, a location in relation to a reference frame such as a reference frame of the display of UI device 106 and/or of the world camera 130) via a lookup table or other suitable indexing method.

A signal generated based on the image data (which is stored in association with the known gaze target), may then be used as input to another device, e.g., may be used to control another device (212).

An example of how the stored image data (or a signal generated based on the stored image data) may be used as input to another device, e.g., for controlling another device, is schematically illustrated in FIG. 2B. In this example, a method for collecting reference images of a person's retina, is performed by processor 102 and includes providing a known gaze target for a person to look at (202) and while the person is looking at the known gaze target, obtaining a plurality of reference images of the person's retina (204), each of the images capturing a different portion of the retina.

Image data from the plurality of reference images, is stored in association with the known gaze target, e.g., in a reference database (206).

The stored image data is then compared with image data of the person's retina while the person is looking at an unknown gaze target (207), to calculate the unknown gaze target (208). An “unknown gaze target” is a target at an unknown location, (e.g., at an unknown location in relation to a frame of reference) and/or at an unknown direction of gaze of the user. Calculating the unknown gaze target may include calculating the unknown location and/or direction of gaze.

The comparison (referred to in step 207) may include, for example, finding a spatial transformation between an image of the retina at a known direction of gaze (a reference image) and a matching image of the retina at an unknown direction of gaze (an input image). In one embodiment, processor 102 receives an input image and compares the input image to a reference image, to find a spatial transformation (e.g., translation and/or rotation) of the input image relative to the reference image. Typically, the comparison includes finding a transformation that optimally matches or overlays the retinal features of the reference image on the retinal features of the input image. A change in orientation of the person's eye, which corresponds to the spatial transformation is then calculated. The direction of gaze associated with the input image can then be determined based on the calculated transformation (or based on the change in eye orientation). A signal based on the change in orientation and/or based on the person's direction of gaze associated with the input image, may then be output (210).

For example, the location of the unknown target and/or a change in rotation of the person's eye caused by looking at the unknown gaze target, may be output (e.g., transmitted) to another device and/or may be displayed and/or used to control another device, such as a device operating based on gaze tracking.

Devices operating based on gaze tracking may include, for example, industrial machines, devices used in sailing, aviation or driving, devices used in advertising, computer games, devices used in entertainment, XR devices (e.g., devices using virtual, augmented or mixed reality), devices used in medical applications, etc. These devices may be controlled based on a person's direction of gaze, determined, for example, as described herein.

In another example, a device used in a medical procedure may include components of the system described in FIG. 1A. For example, a system may include a retinal camera consisting of an image sensor to capture images of different portions of a person's retina. The processor may stitch together the images to create a panorama image of the person's retina. The processor can cause the panorama image to be displayed, e.g., to be viewed by a professional. In some embodiments, the processor can run a machine learning model to predict a health state of the person's eye based on the panorama image, the machine learning model trained on panorama images of retinas of eyes in different health states. Alternatively or in addition, a device for biometric user identification and/or authentication may use the stored image data as a reference database by which to identify specific users.

Collecting reference images and storing reference images in a reference database, such as described herein, may be done at an initial stage (e.g., when enrolling a user to a specific eye tracking system, such as an eye tracking system on XR glasses 110) and/or during later stages, e.g., during use of the system.

In some embodiments, an under-imaged portion of the person's retina may be detected and based on the under-imaged portion, processor 102 may calculate one or more different locations of the gaze target that will require eye rotations and/or angles of rays of gaze that will make the under-imaged portions of the retina, visible to the camera 103. Thus, based on the under-imaged portion, a gaze target may be provided at a different calculated location and images of the person's retina are obtained while the person is looking at the known gaze target at the different location. In another embodiment, based on the under-imaged portion, instructions may be provided to the user (e.g., by processor 102) to move the head, for example, to capture reference images of under-imaged parts of the retina and/or to complete the scan of the retina.

Detecting an under-imaged portion of the retina can be done, for example, by marking an area on a map representing the person's retina, in accordance with a number of images obtained of the area, and detecting in the map a relatively sparsely marked area. The relatively sparsely marked area in the map can be determined to represent the under-imaged portion. Another method for detecting an under-imaged portion of the retina may include stitching a plurality of images together to create a panorama of the person's retina and determining that a missing part of the panorama represents the under-imaged (e.g., unimaged) portion. Stitching images to create a panorama may be done by using standard techniques, such as feature matching and/or finding areas where two images share an area that is very similar (e.g., by Pearson correlation or square difference), merging the two into one image, and repeating until all images are merged to one.

In one embodiment, processor 102 determines that a certain image of the reference images of the person's retina is a false image, namely, a reference image for which it is not (or cannot be) determined that the gaze target associated with the image is the known gaze target. In one example, a false image is captured when the person was looking at a gaze target other than the known gaze target. In another example, a false image is an image with no overlap with other reference images.

When a false image is detected, processor 102 may generate a command to remove image data related to the certain reference image from the reference database or to store the image data related to the certain reference image in a database not used as a reference for tracking gaze of the person.

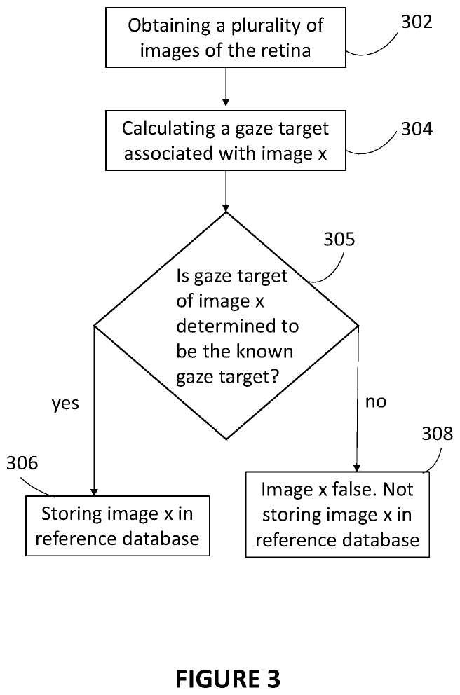

FIG. 3 schematically illustrates an exemplary method for maintaining a reference database of images of a person's retina, the reference database used as a reference for tracking gaze of the person, e.g., as described herein. The method, which may be carried out by processor 102, includes obtaining a plurality of reference images of the person's retina while the person is assumed to be looking at a known gaze target (302). Each of the reference images captures a different portion of the retina. Typically, at least some of the reference images capture partially overlapping different portions of the retina.

A gaze target associated with a certain reference image (e.g., image X) from the plurality of reference images is calculated (304). If the calculated gaze target is determined to be the known gaze target (305), then a command is generated to store image data related to the certain reference image in the reference database (306). If the calculated gaze target is not determined to be the known gaze target (305), then a command is generated to remove the image data related to the certain reference image from the reference database or to store the image data related to the certain reference image in a database not used as a reference for tracking gaze of the person (308).

This process can be done for a second image from the plurality of images obtained in step 302, and so on.

This method ensures that false reference images are not used as a reference for gaze tracking but may be used in other applications. For example, a false reference image may be stored in a database used to create a panorama of a person's retina, as described above.

Typically, image data related to the plurality of reference images is stored in the reference database in association with the known gaze target. Thus, step 306 may include storing image data related to the certain reference image, in association with the known gaze target. Step 308 may include deleting the image data related to the certain reference image from the reference database and/or deleting the image data prior to it being stored in the reference database and/or using the image data for a purpose other than being used as a reference image.

In one example, the calculated gaze target can be determined to be the known gaze target when the calculated gaze target conforms with the known gaze target with a predetermined similarity. The predetermined similarity may be a similarity within a predetermined range, or above or below a predetermined threshold. For example, a predetermined threshold may be a certain degree, or a predetermined range may be a range of degrees. In one example the predetermined range is 0.5-0 degrees, thus, if there is a difference of less than half a degree between the calculated gaze target (e.g., the direction of gaze) and the known gaze target (or an average known target), then the calculated target may be determined to be the known gaze target. If the difference is above 0.5 degrees, then the calculated target may not be determined to be the known gaze target.

Determining whether a calculated gaze target is the known gaze target (as in step 305) may include calculating an actual direction of gaze associated with the certain reference image by comparing the certain reference image to at least one of the plurality of reference images. For example, comparing the certain reference image to several of the plurality of reference images may be done by finding a spatial transformation between the several of the plurality of reference images and the certain reference image and calculating the actual direction of gaze associated with the certain reference image, based on the transformation (e.g., as described above).

An expected direction of gaze associated with the certain reference image is also calculated, based on the known gaze target. A comparison is then preformed between the actual direction of gaze and the expected direction of gaze and the determination of whether the calculated gaze target is the known gaze target, is done based on the comparison.

A high similarity (e.g., a similarity above or below a predetermined threshold or within a predetermined range) may indicate that the target of gaze associated with the certain reference image is indeed the known gaze target. In other cases, the calculated gaze target is not determined to be the known gaze target if it is determined, e.g., based on the similarity, that the gaze target associated with the certain reference image is not the known gaze target.

In some cases, if there is no image found from the plurality of reference images, which at least partially overlaps with the certain reference image, then a comparison of images cannot take place and the calculated gaze target is not (and cannot be) determined to be the known gaze target.

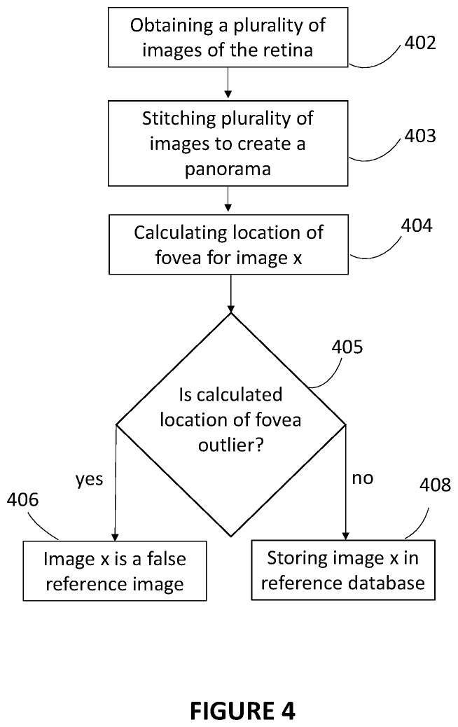

An example of another method for determining whether a calculated gaze target is the known gaze target, is schematically illustrated in FIG. 4. In one embodiment, the method includes obtaining a plurality of reference images of the person's retina while the person is assumed to be looking at a known gaze target (402). Each of the reference images captures a different portion of the retina. Typically, at least some of the reference images capture partially overlapping different portions of the retina.

The obtained images are stitched together (403) to create a panorama image of the person's retina (e.g., as described above). A location of fovea is then calculated for a certain reference image of the plurality of reference images (404). Since the location of the fovea corresponds to the direction of gaze of the person, the location of the fovea can be calculated by using the center of the pupil (which can be detected by using known techniques) as an estimate of the origin of the ray of gaze, and calculating a vector connecting between the center of the pupil and the known gaze target. The direction of this vector can then be mapped to a virtual pixel location (according to the camera properties) which determines the location of the fovea relative to the retinal features in the image.

If (in step 405) a location of fovea calculated for certain image (e.g., image x) is an outlier in relation to other calculated locations of fovea (locations of fovea calculated for other images from the plurality of images making up the panorama) then a gaze target associated with the certain reference image is not determined to be the known gaze target, namely, image x is determined to be a false image (406) and the certain reference image is not saved in or is deleted from the reference database.

If (in step 405) the location of fovea calculated for image x is similar to the other calculated locations of fovea, then image x is stored in the refence database (408). The similarity of locations of fovea may be determined based on their angular location. For example, if a calculated location of fovea for image x is more than half a degree different from the locations of fovea (e.g., from an average location of fovea) calculated for the other images, then image x is determined to be a false image.

The “location of fovea” may refer to an actual pixel, if the fovea is visible in the images, or to a theoretical pixel, if the fovea is not visible in the images. The pixel is the camera's projection of the gaze direction vector on the camera sensor. A theoretical (or calculated) location of the fovea refers to a theoretical pixel that is outside the visible parts of the retina in the image, and can even be (and usually is) outside the camera field of view.

Methods for detecting false images in a reference database, such as described above, may be performed on images of the retina captured while the user is using continuous eye movement (e.g., as described herein) or while the user is looking at differently located targets, using discontinuous eye movement.

Embodiments of the invention ensure that false reference images are not used as a reference for gaze tracking, providing an improved basis for smooth and accurate operation of a gaze tracking system.

1. A method for maintaining a reference database of images of a person's retina, the method comprising:
obtaining a plurality of reference images of the person's retina while the person is assumed to be looking at a known gaze target, each of the reference images capturing a different portion of the retina;
calculating a gaze target associated with a certain reference image from the plurality of reference images;
if the calculated gaze target is determined to be the known gaze target, then generating a command to store image data related to the certain reference image in a reference database used as a reference for tracking gaze of the person; and
if the calculated gaze target is not determined to be the known gaze target, then generating a command not to store the image data related to the certain reference image in the reference database or to remove the image data related to the certain reference image from the reference database.

2. The method of claim 1 wherein at least some of the reference images comprise partially overlapping different portions of the retina.

3. The method of claim 1 comprising determining that the calculated gaze target is the known gaze target when the calculated gaze target conforms with the known gaze target with a predetermined similarity.

4. The method of claim 1 comprising
calculating an actual direction of gaze associated with the certain reference image by comparing the certain reference image to at least one of the plurality of reference images;
calculating an expected direction of gaze associated with the certain reference image based on the known gaze target;
preforming a comparison between the actual direction of gaze and the expected direction of gaze; and
determining whether the calculated gaze target is the known gaze target, based on the comparison.

5. The method of claim 4 wherein comparing the certain reference image to at least one of the plurality of reference images comprises:
finding a spatial transformation between the at least one of the plurality of reference images and the certain reference image; and
calculating the actual direction of gaze associated with the certain reference image, based on the transformation.

6. The method of claim 1 wherein if no image from the plurality of reference images that at least partially overlaps with the certain reference image, is found, then the calculated gaze target is not determined to be the known gaze target.

7. The method of claim 1 comprising:
creating a panorama image of the person's retina from the plurality of reference images;
calculating a location of fovea for the certain reference image; and
if the calculated location of fovea is an outlier in relation to other calculated locations of fovea, then a gaze target associated with the certain reference image is not determined to be the known gaze target.

8. The method of claim 1 comprising storing in the reference database image data related to the plurality of reference images in association with the known gaze target.

9. The method of claim 1 comprising using the reference database as a reference for tracking gaze of the person by comparing information from the reference database with image data of the person's retina while the person is looking at an unknown gaze target, to calculate the unknown gaze target.

10. The method of claim 9 comprising using a signal generated based on the calculated unknown gaze target to control a device.

11. The method of claim 10 wherein the device comprises an XR device.

12. The method of claim 1 comprising:
detecting an under-imaged portion of the person's retina;
changing a location of the known gaze target in accordance with the under-imaged portion; and
obtaining images of the person's retina while the person is looking at the known gaze target after changing the location.

13. The method of claim 1 comprising:
detecting an under-imaged portion of the person's retina;
based on the under-imaged portion, providing instructions relating to movement of the person's head; and
obtaining images of the person's retina while the person is looking at the known gaze target while moving the person's head according to the instructions.

14. The method of claim 1 comprising displaying the known gaze target on a display of a user interface (UI) device.

15. A gaze tracking system comprising:
a camera to capture images of a person's retina while the person is assumed to be looking at a known gaze target;
a reference database to store image data related to the images of the person's retina in association with the known gaze target; and
a processor to:
calculate a gaze target associated with a certain image from the images of the person's retina;
if the calculated gaze target is determined to be the known gaze target, then store image data related to the certain image in the reference database; and
if the calculated gaze target is not determined to be the known gaze target, then not to store image data related to the certain image in the reference database.

16. The system of claim 15 wherein the processor is to compare information from the reference database, with image information of the person's retina while the person is looking at an unknown gaze target, to calculate a location of the unknown gaze target.

17. The system of claim 15 comprising a user interface (UI) device configured to display the known gaze target.

18. The system of claim 17 wherein the known gaze target is a moving target.

19. The system of claim 15 comprising an XR device operative based on the reference database.
